(19) United States
(12) Patent Application Publication
(10) Pub. No.: US 2023/0033101 A1
(43) Pub. Date: Feb. 2, 2023

(54) METHOD AND DEVICE FOR ANALYSIS OF LIQUID SAMPLES

(71) Applicant: QLIFE APS, Ballerup (DK)

(72) Inventors: Peter WARTHOE, CopenhangenØ (DK); Ebbe FINDING, Gentofte (DK); Robert ELKÆR, Birkerød (DK)

(73) Assignee: QLIFE APS, Ballerup (DK)

(21) Appl. No.: 17/785,517

(22) PCT Filed: Dec. 21, 2020
(86) PCT No.: PCT/EP2020/087539

§ 371 Date: Jun. 15, 2022

(30) Foreign Application Priority Data
Dec. 31, 2019 (DK) PA 2019 01564

(51) Int. Cl. G01N 1/40; G01N 1/38; G01N 33/72; G01N 21/31; G01N 21/64

(52) U.S. Cl. CPC G01N 1/4005; G01N 1/38; G01N 33/721; G01N 21/31; G01N 21/64; G01N 2001/4016; G01N 2001/388

(57) ABSTRACT

The present invention relates to a method and a device for quantitatively detecting the presence or absence of an analyte in a liquid sample, comprising the steps of providing a set of parts comprising a container for collecting a liquid sample material, and a filter material and a detection device comprising a reaction liquid, thereafter adding a metered amount of liquid sample material to the container, thereafter transferring the metered amount of liquid sample material from the container to the filter material, thereafter contacting the filter material containing the metered amount of sample material with the reaction liquid and mixing the reaction liquid and the filter material, thereby obtaining a detection liquid, thereafter measuring the transmission of electromagnetic radiation at one or more wavelengths through the detection liquid and/or the emission of electromagnetic radiation at one or more wavelengths from the detection liquid and detecting the amount of analyte in the sample by comparing the results obtained in step e. with an internal standard, the method being characterised in that the metered amount of sample is transferred from the container to the filter material by use of capillary forces.

### TECHNICAL FIELD

The present invention relates to methods and a device for quantitatively detecting the presence or absence of an analyte in a liquid sample with improved sensitivity, precision, and total assay time. In particular, the present invention relates to a method and a kit-of-parts for quantitatively detecting the presence or absence of a biomarker in a blood sample.

### BACKGROUND

Many diseases may be monitored by monitoring the presence or absence of a particular analytes, such as markers or biomarkers, in body fluid samples, particularly by monitoring the presence or absence of particular analytes in blood samples.

When monitoring such disease markers, the sensitivity, precision, and total assay time (TAT) of the apparatuses and methods used for analysis are always important issues.

In a point-of-care apparatus and a method for analysing a liquid such as blood, it is not practically acceptable to use sample sizes of more than 50 μl. Often, the sample size is restricted to the amount of liquid being present in 1-2 drop(s) of blood, i.e. approx. 40 μl or 20 μl, or less. When working with such small volumes of samples, the sensitivity, precision and TAT of the apparatuses and the methods used for analysis become highly significant issues, and ways of increasing the sensitivity, precision, and/or TAT of the apparatuses and methods are always challenging.

Previously, when analysing a small amount of a liquid sample, such as a drop of blood, improvements in sensitivity, precision and TAT have been achieved in a range of different ways.

Some methods and apparatuses focus on providing improvements with respect to accurately metering a sample, whereby increased precision may be obtained. The technique involves the use of a sampler with a porous hydrophilic tip that enables the collection of small, accurate and precise blood volumes. The collection process usually takes around 2-4 seconds regardless of the HCT level. After drying, the samples can be stored, transported or directly analysed. The technique is gaining more and more attention because of its simplicity and cost effectiveness. The purpose of the technique is to improve test reliability by providing fixed volume sample of blood and facilitate self-sampling with minimal instructions.

Others focus on providing improved methods and apparatuses capable of accurately mixing the constituents of a particular sample and the detection means (fluorophores, transmission, absorbance etc.), whereby increased sensitivity and precision may be obtained.

Other methods focus on improving the quality of the sample material (e.g. by removing undesired constituents of the sample) used for the analysis. However, accurate analysis of specific analytes present in liquid samples represents a ubiquitous problem in the art, especially for methods and apparatuses for point-of-care home applications.

Accordingly, one object of the present invention is to improve the sensitivity, precision and TAT of existing devices and methods based on optical measurements that are capable of quantitatively detecting the presence or absence of one or more analytes in a liquid sample, such as a liquid sample comprising less than 50 μL.

Optically based methods are to be understood as methods relying on an optical measuring system where a source of electromagnetic radiation irradiates a liquid sample present in a container (e.g. a cuvette), whereafter the absorption and/or emission of electromagnetic radiation from the sample in the cuvette is monitored.

### BRIEF DESCRIPTION OF THE INVENTION

One important improved modification according to the present invention is that the metering and introduction of sample entering the analytical procedure are exclusively performed by use of capillary forces.

The inventors experienced that any step of metering sample material manually and applying the sample by use of external force in a point-of-care setting will inevitably lead to an increase in assay inaccuracy and be problematic for providing precise and reliable assay results. This problem was solved by metering the sample material exclusively by capillary forces.

In a highly preferred embodiment of the invention, the metering was performed by collecting the metered amount of liquid sample by use of a container that was capable of containing and collecting a metered amount of sample from a larger liquid sample by capillary forces and subsequently contacting the sample-filled container with a filter material capable of containing at least the metered amount of sample, and subsequently contacting the filter material containing the sample with the analytical reaction liquid.

Accordingly, in one highly preferred aspect, the invention relates to a method for measuring the amount of an analyte in a liquid sample, the method comprising the steps of:
a. providing a set of parts comprising
   i. a container for collecting a liquid sample material,
   ii. a filter material
   iii. a detection device comprising a reaction liquid,
b. adding a metered amount of liquid sample material to the container,
c. transferring the metered amount of liquid sample material from the container to the filter material,
d. contacting the filter material containing the metered amount of sample material with the reaction liquid and mixing the reaction liquid and the filter material, thereby obtaining a detection liquid,
e. measuring the transmission of electromagnetic radiation at one or more wavelengths through the detection liquid and/or the emission of electromagnetic radiation at one or more wavelengths from the detection liquid,
f. detecting the amount of analyte in the sample by comparing the results obtained in step e. with an internal standard,
   the method being characterised in that the metered amount of sample is transferred from the container to the filter material in step c by use of capillary forces.

This invention is embodied in the Egoo device capsule used below in the examples. The technical benefits of the invention are apparent from the examples below.

### DEFINITIONS

In this invention the word “filter” and “membrane” are used as synonyms. In the context of the present invention, a filter material means any commercially available filter (membrane) material, such as Fusion 5 or Whatman903, or any other a hydrophilic filter material capable of containing and passively withholding a certain amount of liquid material, and further capable of separating a liquid sample into a liquid phase (such as plasma or serum) and a retentate phase (such as blood cells), i.e. withholding certain components of a particular sample (e.g. blood cells, cell membrane components or high molecular weight substances). A preferred filter material is “Fusion 5”, which is a single layer matrix membrane filter, that can be used to replace traditional modular components from a lateral flow testing kit.

Preferably, the filter material is used as a flat circular disc with a diameter of less than 50 mm. Even more preferred, the diameter of the filter material according to the invention is less than 10 mm, such as less than 5 mm, or less than 3 mm or even less than 2 mm. In a most preferred aspect, the filter material has a diameter of 5 mm or less.

In this invention, the word “container” is meant to comprise a compartment capable of containing a liquid sample, such as a tube or a pipette or the like. Preferably, the container is a hollow cylinder capable of withdrawing a metered amount of sample by capillary force.

### Precision

The precision of an analytical procedure expresses the closeness of agreement (degree of scatter) between a series of measurements obtained from multiple sampling of the homogeneous sample under the prescribed conditions. Precision may be considered at three levels: 1) repeatability, 2) intermediate precision, and 3) reproducibility. Repeatability expresses the precision under the same operating conditions over a short interval of time. Repeatability is also termed intra-assay precision. Intermediate precision expresses within-laboratories variations: different days, different analysts, different equipment, etc. Reproducibility expresses the precision between laboratories (collaborative studies usually applied to standardization of methodology). Precision should be investigated using homogeneous, authentic (full scale) samples. However, if it is not possible to obtain a full scale sample, it may be investigated using a pilot-scale or bench-top scale sample or sample solution. The precision of an analytical procedure is usually expressed as the variance, standard deviation or coefficient of variation of a series of measurements.

### Accuracy

Accuracy is a description of systematic errors, a measure of statistical bias, as these cause a difference between a result and a “true”value. In simplest terms, given a set of data points from repeated measurements of the same quantity, the set can be said to be precise if the values are close to each other, while the set can be said to be accurate if their average is close to the true value of the quantity being measured. The concepts of accuracy and precision are independent of each other, so a particular set of data can be said to be either accurate or precise, both accurate and precise, or neither accurate nor precise.

Metering of samples

Conventionally, the precise metering of liquid samples and the precise addition thereof to an assay reagent is performed by laboratory technicians in a clinical setting using clinical pipetting equipment. This is, however, unfeasible in a point-of-care setting. Accordingly, one-time use pipettes have been designed and marketed that are capable of withdrawing and containing a precise sample volume from a larger source by capillary forces. Such pipettes are designed to release sample when the user presses the bulk end of the pipette while simultaneously blocking an airhole therein, thereby forcing the metered liquid sample out of the pipette.

However, during the research leading to the present invention, it was discovered that active handling of these pipettes (e.g. handling according to the intended use, wherein the user presses the bulk end of the pipette while simultaneously blocking an airhole) resulted in severe inaccuracies of the measurements of the provided metered sample.

Therefore, it was discovered that it was necessary to retract sample material from the pipettes also by capillary forces only. This was achieved by introducing a filter material withdrawing the metered amount of sample from the metering pipette, whereafter the filter material containing the sample was subjected to further assay manipulations.

### DETAILED DISCLOSURE OF THE INVENTION

In one aspect of the invention, it was surprisingly found that adding the liquid sample material to the detection assay by adding a filter material containing a metered amount of sample directly to the detection liquid (or contacting it with the analytic liquid) produced significantly superior results in terms of both accuracy and precision.

Further, it was found possible to add a precisely metered amount of sample while at the same time providing a desired purification of the sample, as the filter material is capable of withholding certain sample contaminants.

Thus, the gist of the present invention relies on metering the sample and transferring the sample between assay compartments by capillary forces.

In one embodiment of the invention, as performed by the Egoo detection system described below, a filter (containing the metered sample) is brought into contact with the liquid in the main cuvette of the capsule, and the sample constituents are simply removed from the filter by oscillating mixing actions, whereby the filter material empties the sample constituents producing the detection liquid. In the Egoo capsule, the filter material is brought into contact with the liquid in the main cuvette by penetrating a foal sealing separation the respective compartments, whereafter the sample material contained in the filter material is released into the assay solution by vortexing the detection liquid, whereby the entire metered amount of sample is released producing the detection liquid.

Thus, in one aspect the present invention relates to a method for measuring the amount of an analyte in a liquid sample, the method comprising the steps of:
a. providing a set of parts comprising
   i. a container (e.g. a pipette capable of metering a precise volume of liquid sample) for collecting sample material, and
   ii. a filter material
   iii. a detection device comprising a reaction liquid,
b. adding a metered amount of liquid sample material to the container,
c. transferring a metered amount of sample from the container to the filter material,
d. contacting the filter material containing the metered sample material with the reaction liquid and mixing the liquid and the filter material, thereby providing a detection liquid,
e. measuring the transmission of electromagnetic radiation at one or more wavelengths through the detection liquid and/or the emission of electromagnetic radiation at one or more wavelengths from the detection liquid,
f. detecting the amount of analyte in the sample by comparing the results obtained in step e. with an internal standard,
   the method being characterised in that the metered amount of sample is transferred from the container to the filter material in step c by use of capillary forces only.

It was initially speculated that precise metering could be obtained by use of a filter material capable of containing only the precise metered amount of sample material. This was, however, found not to be feasible. In contrast, it was found that it was necessary to have excess capacity in the filter material, and to perform metering of sample by means of the metering container. Therefore, in a highly preferred aspect of the invention, the filter material is capable of containing more liquid than the metered amount of liquid sample material added to the container in step b.

It was also surprisingly found that efficient and accurate transfer of liquid sample material from the container to the filter was enhanced by the filter material being positioned such that the fibres in the filter material run parallel to the flow direction of the liquid sample out of the container.

Further, in one embodiment, the invention comprises a kit-of-parts for performing the above method. Such kit of parts comprises:
i. a container for collecting a metered amount of liquid sample material by capillary forces,
ii. a filter material capable of collecting the metered amount of liquid sample material from the container by capillary forces, and
iii. a detection device comprising a reaction liquid.

The kit-of-parts preferably also comprises
iv. a detection assembly comprising a source of electromagnetic radiation, means for detecting electromagnetic radiation, and means for receiving a detection device comprising a liquid sample,
v. a means for providing rapidly oscillations in a circular or ellipse motion of the detection assembly.

Any optical method may ultimately be used to detect the presence of analytes in samples according to the inventions described herein. These include spectroscopic and spectrophotometric methods of analysis. The use of spectrophotometers spans various scientific fields, such as physics, materials science, chemistry, biochemistry, and molecular biology.

Spectroscopy and spectrophotometry are conventionally used for quantitative measurement of the absorption, reflection and/or transmission properties of a material (an analyte) as a function of wavelength of light absorbed/emitted from the sample. The use of these techniques is well known in the art.

Absorption spectroscopy refers to spectroscopic techniques that measure the absorption of radiation, as a function of frequency or wavelength, due to its interaction with a sample. The sample absorbs energy, i.e., photons, from the radiating field. The intensity of the absorption varies as a function of frequency, and this variation is the absorption spectrum. Absorption spectroscopy is performed across the electromagnetic spectrum.

Absorbance spectroscopy, commonly referred to as spectrophotometry, is the analytical technique based on measuring the amount of light absorbed by a sample at a given wavelength. Spectrophotometry, particularly in the visible and UV portions of the electromagnetic spectrum, is one of the most versatile and widely used techniques in chemistry and the life sciences. Absorption spectroscopy is employed as an analytical chemistry tool to determine the presence of a particular substance in a sample and, in many cases, to quantify the amount of the substance present. Infrared and ultraviolet-visible spectroscopy are particularly common in analytical applications. Absorption spectroscopy is also employed in studies of molecular and atomic physics, astronomical spectroscopy and remote sensing.

There is a wide range of experimental approaches for measuring absorption spectra. The most common arrangement is to direct a generated beam of radiation at a sample and detect the intensity of the radiation that passes through it. The transmitted energy can be used to calculate the absorption. The source, sample arrangement and detection technique vary significantly depending on the frequency range and the purpose of the experiment.

Fluorescence spectrometry is a fast, simple, and inexpensive method to determine the concentration of an analyte in a solution based on fluorescent properties. It can be used for relatively simple analyses, where the type of compound to be analyzed (the analyte) is known, e.g. to perform a quantitative analysis to determine the concentration of the analyte in the samples. Fluorescence is used mainly for measuring compounds in a solution.

In fluorescence spectroscopy, an electromagnetic beam passes through a solution in a cuvette and an analyte in the sample absorbs energy from the beam. This energy is emitted as an electromagnetic beam (light) with a different wavelength. The amount of light that is absorbed and emitted by the sample is proportional to the presence of analyte in the sample. In fluorescence spectrometry, both the excitation spectrum (the light that is absorbed by the analyte) and/or an emission spectrum (the light emitted by the exited analyte) can be measured. The concentration of the analyte is directly proportional with the intensity of the emission.

Turbidimetry is the process of measuring the loss of intensity of transmitted light due to the scattering effect of particles suspended in it. Light is passed through a filter creating a light of known wavelength which is then passed through a cuvette containing an assay solution. A photoelectric detector collects the light which passes through the cuvette. A measurement is then given for the amount of absorbed light.

Immunoturbidimetry is an important tool in the broad diagnostic field of clinical chemistry. It is used to determine proteins not detectable with classical clinical chemistry methods. Immunoturbidimetry uses the classical antigen-antibody reaction. The antigen-antibody complexes are particles which can be optically detected by a photometer. In more detail, liquid sample is added to a buffer solution and mixed with a suspension of monoclonal antibody against analyte that is bound to latex. The analyte binds to the latex-bound antibody and agglutinates. The light scattering caused by the increase in particle size is used as a measure of analyte concentration. The amount of light scattering is proportional to the concentration of analyte in the sample.

### General Procedures when Performing Optical Detection Methods

Conventionally, optical detection methods rely on the introduction of a liquid sample directly into a container (e.g. a “cuvette”) and the measurement of the change in an optical signal generated by the presence of the sample. Normally, the container contains a sample-free liquid prior to the introduction of the sample. The reaction liquid may contain certain reagents that may interact with the analyte in the sample to produce a signal in the presence of the analyte. Alternatively, such reagents are added after introduction of the sample. The container may e.g. in certain methods contain a fluorophore, which upon the arrival of a liquid in the container may be solubilised.

A sample blank measurement may be performed to provide a background reference. The background measurement is performed in order to correct the sample measurements for unspecific signal (“noise”), which is signal generated by other constituents than the analyte in the detection liquid as well as the influence of the system (e.g. the container) on the signal. Unspecific signals could be generated, e.g. by blood haemolysis effecting the quality of filtrated plasma/serum. Thus, the generalised methods contain steps of providing a sample blank measurement by measuring the transmission and/or emission of electromagnetic radiation at one or more wavelengths through the first liquid at time T₀.

In this respect, the To measurements is measured prior to the introduction of the sample or, alternatively, prior to (or immediately following) introducing the reagent providing a quantitative change in the transmission/emission of radiation from the sample and providing the signal generated by the background in the sample. Repeated measurements may be performed in order to increase the precision of the blank measurement.

The introduced sample/reagent alters the transmission or emission of electromagnetic radiation at one or more wavelengths through the detection liquid, and the degree of alteration reflects the degree of presence of the analyte in the introduced sample. In other words, the introduction of sample/reagent produces an alteration in a detectable radiation-based signal, and the alteration is quantitatively proportional to the amount of sample present (e.g. determined by use of internal standards with known concentration of sample).

After introducing the sample to the reagents, the constituents of the generated detection liquid must be mixed thoroughly in order to generate an accurate and precise sample measurement.

### PREFERRED EMBODIMENTS OF THE INVENTION

In one embodiment, the invention is performed with an Egoo device as described in more detail below.

### Egoo Device

The Egoo device is a Micro Opto Electro Mechanical device capable of executing the present invention. The entire Egoo device consists of an optical unit and an Egoo capsule for the measurement of biomarkers in human blood.

The disposable assay capsules contain all assay reagents for performing an assay. The assay capsules are inserted into Egoo device, and the assay is then run automatedly.

Essentially, the Egoo device consists of a detection assembly consisting of a source of light and a detector situated such that an assay cuvette can be placed in between the source of light and the detector. The Egoo device further comprises means for vortexing the entire detection assembly when an Egoo capsule is added to the assembly. The Egoo device is supplemented with a capsule comprising an assay cuvette and a separate chamber comprising a filter material on which liquid sample may be added.

Thus, the Egoo device is a kit-of-parts designed for point-of-care use by unexperienced users.

The kit-of-parts comprises a measuring device and a capsule for receiving sample material.

### Egoo Optical Unit

The Egoo optical unit comprises a conventional optical measuring system, comprising a “detection assembly” comprising a source of electromagnetic radiation, a means for detecting electromagnetic radiation and a means for receiving a container (“cuvette”) comprising a liquid, said means and container being positioned between the source and the detection means such that electromagnetic radiation radiates through the liquid from the source to the detection means. After receipt of the container comprising a sample liquid, the device is capable of subjecting the entire optical measuring system (the detection assembly) to oscillating motions (“vortex”), as opposed to conventional devices and methods, wherein oscillations of only the container comprising the liquid are standard.

The optical system located inside the detection assembly consists of two optical paths. In optical path 1, the transmission is measured at 570 nm using the LED 570 nm as light source and the photodiode 1 as detector measuring the absorbance signal. In optical path 2, the light source is the LED 390 and the photodiode 1 is the detector for measuring the fluorescence signal.

### Egoo Capsule

The Egoo capsule comprises a main cuvette (sometimes referred to as the reaction chamber) as well as separated compartments.

One compartment (compartment 1) comprises a hydrophilic filter material capable of containing (at least) an amount of liquid corresponding to the metered amount of sample material.

Other compartments are fluid-filled containers containing assay reagents (compartment 2-4). Further, the capsule contains plungers/seal breakers which can be activated such that reagents or material from each compartment may be in fluid communication with the liquid in the main cuvette after breakage of a liquid impermeable seal and/or enters the main cuvette by being injected down through the sealing into the cuvette.

### Sample Material

Preferably, the liquid sample is a sample consisting of less than 40 μl of liquid. Such sample size is relevant for automated methods and apparatuses. More preferably, the liquid sample is a sample consisting of less than 20 μl of liquid. Such sample size is relevant for point-of-care apparatuses and methods.

In a highly preferred embodiment, the sample to be analysed is a blood sample. In one preferred embodiment, the blood sample is whole blood. In another preferred embodiment, the blood sample is a blood plasma sample.

### Mixing

In the above-mentioned methods, it is highly preferred that the mixing of the contents of the detection liquid is performed by rapidly oscillating the detection liquid ion in a circular of ellipse motion at a speed of at least 1000 rpm. Preferably, the mixing is performed by rapidly (1000-4000 rpm) oscillating the detection liquid in a circular of ellipse motion (vortexing).

### Source of Electromagnetic Radiation

The detection assembly comprises a source of electromagnetic radiation, a source of electromagnetic radiation being defined as a means from which electromagnetic radiation is emitted. The relevant electromagnetic radiation may in principle be of any suitable wavelength. However, electromagnetic radiation in the wavelength between 300 nm and 900 nm is preferred.

### Means for Detecting Electromagnetic Radiation

The analyte detection assembly comprises a means for detecting electromagnetic radiation, a means for detecting electromagnetic radiation being defined as a means with which electromagnetic radiation is detected (i.e. absorbed and converted into electrical energy). The relevant electromagnetic radiation to be detected may in principle be of any suitable wavelength. However, the electromagnetic radiation to be detected must be suitable in view of the electromagnetic radiation being emitted by the source and/or by the sample.

### Analytes

In general, the methods and apparatuses of the invention can be used to measure all blood biomarkers within clinical chemistry, cancer diagnostics and all other related diagnostics fields.

The methods and apparatuses of the inventions are, however, preferably used for the detection of one or more of the following blood markers (analytes); Phenylalanine (phenylketonuria patients), CRP, hs-CRP, Lipid Panel (inflammation and cardiovascular vascular disease biomarkers), Lipid profile (total cholesterol, HDL and Triglyceride), HbA1c (diabetes biomarkers), ALAT (liver biomarker), Vitamin D and D-dimer.

In a preferred embodiment of the invention, the reaction liquid comprises a substance which binds to an analyte present in the sample, such as the fluorophore eosin-borate-acid for HbA1 detection.

### EXAMPLES

The purpose of the examples below is to describe and compare the assay precision, % CV (standard deviation/mean * 100) using the described invention.

In example 1, five different methods—one of which is a method according to the invention—are tested in a precision study for metering and collection of a sample.

In example 2, the precision of a phenylalanine assay on a blood sample is tested using either the described invention vs. the standard method of today for collection and analysis of phenylalanine in blood samples.

In example 3, the precision of a haemoglobin assay on a blood sample is tested using either the described invention vs. alternative ways of performing the metering and blood collection process.

### The Invention may be Performed on an Egoo Device

Essentially, the Egoo device consists of a detection assembly consisting of a source of light and a detector situated such that an assay cuvette can be placed in between the source of light and the detector. The Egoo device further comprises means for vortexing the entire detection assembly when an Egoo capsule is added to the assembly. The Egoo device is supplemented with a capsule comprising an assay cuvette and a separate chamber comprising a filter material on which liquid sample may be added. The filter material (and reagents) may be transferred from the separate chamber to the assay cuvette during operation of the Egoo device, whereby sample material and reagents can be assayed for absorption and/or emission of electromagnetic radiation during operation of the device.

More precisely, the Egoo capsule consists of a sample injector compartment R1, fluid chambers R2, R3 and main cuvette R4. The Egoo device may add the constituents of R1, R2 and/or R3 to the main cuvette (R4) depending on the relevant assay.

### Example 1. A Precision Study for Metering and Collection of a Sample

To explore the described invention, five users were instructed to perform a sample collection and metering process in five different ways. Each metering process was repeated ten times by the user. A blue dye was used as sample material. The membrane used was Porex R34436 which consists of a mixture of polyethylene and polypropylene material.

TABLE 1
Five metering and collection methods

| Method no. | Four metering and collection methods |
|---|---|
| 1 (reference | 15 ul of blue dye sample material was collected and metered using the disposable |
| example) | pipettes described below. The user collected the 15 μl sample by blocking the hole |
| | in the bulk part of the pipette with the fingers and applying pressure on the small |
| | bulk at the end of the pipette, followed by sample contact and release of pressure |
| | until 15 μl of sample had entered into the pipette. |
| | The entire sample was thereafter added from the pipette directly to the detection |
| | liquid in the assay cuvette in an Egoo capsule by blocking the hole in the bulk part |
| | of the pipette with the fingers and applying pressure on the small bulk at the end |
| | of the sample filled pipette. The amount of dye added was analysed. |
| 2 (reference | 15 ul of blue dye sample material was collected and metered using the disposable |
| example) | pipettes described below. The user collected the 15 μl sample by contacting the |
| | end of the pipette to the sample whereby 15 μl of the blue dye enter into the pipette |
| | by capillary forces. |
| | The entire sample was thereafter added from the pipette directly to the detection |
| | liquid in the assay cuvette in an Egoo capsule by blocking the hole in the bulk part |
| | of the pipette with the fingers and applying pressure on the small bulk at the end |
| | of the sample filled pipette. The amount of dye added was analysed. |
| 3 | 15 ul of blue dye sample material was collected and metered using the disposable |
| (embodiment | pipettes described below. The user collected the 15 μl sample by contacting the |
| of the | end of the pipette to the sample whereby 15 μl of the blue dye enter into the pipette |
| invention) | by capillary forces. |
| | The tip of the filled pipette was contacted to a membrane material (Porex R34436) |
| | whereby the entire sample flowed out of the pipette and into the membrane by |
| | passive capillary forces. The sample filled membrane was added to the assay |
| | cuvette in the Egoo capsule and the amount of dye analysed. |
| 4 (reference | 15 ul of blue dye sample material was metered and directly added to a membrane |
| example- | material using a standard calibrated pipette. The sample filled membrane was |
| performed | added to the assay cuvette in the Egoo capsule and the amount of dye analysed. |
| only by a | |
| laboratory | |
| technician) | |
| 5 (reference | 15 ul of blue dye was metered and directly added to the assay cuvette in the Egoo |
| example- | capsule bypassing the collection membrane using a standard calibrated pipette and |
| performed | the amount of dye analysed. |
| only by a | |
| laboratory | |
| technician) | |

### Reagents and Materials Used

### The Egoo Capsule

The Egoo capsule consists of a sample injector compartment R1, fluid chambers R2, R3 and main cuvette R4.

The Egoo device may add the constituents of R1, R2 and/or R3 to the main cuvette (R4) depending on the relevant assay.

### Metering Pipette

15 μl pts collection capillary tubes (CE mark) were used. These pipettes are disposable pipettes designed for collecting and transferring 15 μl sample. The pipettes consist of a capillary tube containing a capillary stop at 15 μl, and a small bulk on the end of the pipette which is pressed (while blocking an air hole) to release the collected sample. The bulk part on the end of the pipette further contains a small hole allowing air to escape, whereby capillary forces may drag 15 μl sample into the pipette (until the capillary stop). During intended use, the user collects 15 μl sample by contacting the sample with the end of the pipette whereby 15 μl of the blue dye enter the pipette by capillary forces. Thereafter, during intended use, the user releases the sample by blocking the hole in the bulk part of the pipette with a finger and pressing the bulk part of the pipette.

### Dye

Bromophenol Blue solution (0.04 wt in water. Sigma-Aldrich 313744 Lot MKCD9662)

### Test Procedure:

1. 15 μl of blue dye was added to the the blood metering transfer pipette described in Table 1 above
2. Each person performed each method 10 times within 1 hour
3. A sample blank measurement was taken by measuring the absorbance to time T_{0.}
4. 15 μl of bromophenol blue solution (0.04 wt in water. Sigma-Aldrich 313744 Lot MKCD9662) was added to the Egoo PHE capsule by the five different procedures outlined in Table 1
5. The blue dye was mixed with the R1 reagent using an oscillating (vortex) movement.
6. At T₁, absorbance measurement was taken, and the results were calculated as absorbance=2−log (T₁/T₀.×100)
7. The % CV was calculated as the standard deviation/mean*100.

TABLE 2
The precision result where each person performed the three different
methods. Since method 4 and 5 are unsuited for point-of-care analysis
and require some training in using a pipette, only the laboratory
technician performed this method.
Precision % CV, n = 10

| | Method 1 | Method 2 | Method 3 | Method 4 | Method 5 |
|---|---|---|---|---|---|
| Person 1 | 53.3 | 11.5 | 2.9 | | |
| Male, age 66 | | | | | |
| Person 2 | 72.4 | 13.5 | 2.5 | | |
| Female, age 29 | | | | | |
| Person 3 | 109.4 | 17.8 | 1.9 | | |
| Male, age 55 | | | | | |
| Person 4 | 204.5 | 24.4 | 3.1 | | |
| Male, age 52 | | | | | |
| Person 5 | 20.5 | 9.8 | 3.2 | 2.9 | 2.8 |
| Laboratory | | | | | |
| Technician | | | | | |

### Discussion

The purpose of example 1 was to explore the possibility of transferring a metered amount of blue dye to the collection membrane on the Egoo capsule using the described invention and to compare the described invention with four other methods.

A low % CV value indicates that the measured values tend to be close to the mean (also called the expected value) of the data set, while a high % CV value indicates that the values are spread out over a wider range.

As can be observed, the precision using method 3 according to the invention (by an untrained point-of-care user) is comparable with the precision obtained by a trained laboratory technician using a calibrated pipette (methods 4 and 5). It can also be observed that applying any kind of active pressure to the transfer pipette (methods 1 and 2) resulted in significantly increased % CV values when performed by point-of-care users, and substantially increased % CV values were also observed even when the procedures (methods 1 and 2) were performed by a skilled laboratory technician.

### Example 2. Comparing the Precision of the Sample Metering and Transfer Method According to the Invention with Today's Standard Method for Metering and Transfer of Blood Samples in a Phenylalanine Assay

The purpose of example 2 was to explore the possibility of integrating a well-known fluorescence-based phenylalanine (PHE) assay together with the described invention and to compare this setup to the standard method of collection and metering of a home-collected blood sample.

Phenylketonuria (PKU) is autosomal recessive genetic disorder caused by a deficiency of hepatic phenylalanine hydroxylase (PAH) activity. In the Caucasian population, about 1 in 50 are carriers, and 1 in 10.000 are affected with PKU. Because of the PAH deficiency, phenylalanine is not converted to the amino acid tyrosine. This causes an excessive amount of PHE and toxic metabolites to accumulate in all parts of the body, including the brain, in blood, and in urine. Those excesses create a chemical imbalance that results in various degrees of mental retardation. During the last decade, several vendors have tested various assay methods to identify the best method for measuring the PHE levels at home, methods that must be comparable to measuring blood sugar at home for people with diabetes. Unfortunately, the phenylalanine molecule is present in 500-1000× lower concentration in blood (μM) compared to glucose (mM). So far, all attempts to identify home-based methods have been struggling with assay-related parameters, such as assay sensitivity, assay precision, assay stability, assay comparison and assay complexity. The present invention has succeeded in overcoming the above assay-related challenges.

Using the present invention, it would be possible to perform such PHE assay in the small Egoo POC device in the users' home having identical or even better analytical performance compared to the state-of-the-art laboratory-based equipment.

### PHE Assay Principle

The Phenylalanine assay makes use of a fluorescence ninhydrin assay method. The assay procedure is a modification of the fluorometric assay procedure first publish by McCaman and Robin, Lab Clin. Med 59, page 885-890 in 1962. The assay is based on a chemical method intended for the quantitative determination of PHE in blood.

### Summary of the PHE Assay Procedure According to the Invention

A precise volume of capillary blood (15 μl) was transferred from a finger to the blood metering transfer pipette as described in example 1, method 3. The blood metering transfer pipette was inserted into the capsule inlet of an Egoo capsule, where it was brought into contact with a membrane material (Whatman-903). When the metering transfer pipette got into physical contact with the membrane material, the blood was passively flowing from the capillary channel in the pipette into the membrane material. After a drying period, the membrane was injected into the main cuvette where the amino acid phenylalanine (and all other amino acids) was extracted out of the membrane by use of an extracting solution (R1) and an oscillating (vortex) movement of the cuvette inside the Egoo device. Next, the R2 reagent was injected into the main cuvette and mixed. After incubating at 48° C. (45-80° C.), the PHE now formed a fluorescence compound with ninhydrin. The fluorometric response and specificity were greatly enhanced by the presence of a dipeptide L-leucyl-L-alanine. The pH during the reaction was strictly controlled by a succinate buffer at 5.8+/−0.1 to maximize specificity. After the ninhydrin reaction, the pH was adjusted to >8.0 for optimal fluorescence detection by injecting the R3 solution into the main cuvette. The fluorescence molecule was measured at 450 nm with the excitation wavelength being 390 nm.

### PHE Assay Reagents:

R1: sample injector containing the membrane

R2: 70 mM Ninhydrin in water.

R3: 0.3 M Na2HPO4, 0.05M NaOH, pH=11.5.

R4: 70% Ethanol, 0.2M Succinate buffer pH 4.9, 0.4% NaCl, 10 mM L-leucyl-L-alanine.

The Egoo capsule consists of a sample injector compartment R1, fluid chambers R2, R3 and main cuvette R4.

The Egoo device may add the constituents of R1, R2 and/or R3 to the main cuvette (R4) depending on the relevant assay.

Procedure 1. The blood metering and collection described in this invention. After metering and adding the blood to the assay capsule, all assay steps were performed by the Egoo device.
1. 15 μl of full blood was added to the the blood metering transfer pipette as described in example 1, method 3
2. The metering transfer pipette was inserted into the inlet of the Egoo PHE capsule
3. When the metering transfer pipette got into physical contact with the Whatman903 membrane material in R1, the blood was passively flowing into the membrane material as described in example 1, method
3. After the blood transfer, the metering transfer pipette was discarded
4. After blood transfer to the Whatman-903 membrane, the filter was dried for 3 hours
5. After drying, the blood membrane from R1 was injected into the main cuvette where the extracting solution (R4) combined with an oscillating (vortex) movement were extracting (releasing) PHE molecules (and other amino acids) into the extracting solution (R4).
6. The R2 reagent was injected into the main cuvette and the fluorescence assay was initiated
7. A sample blank measurement by measuring the fluorescence to time T₀ was taken
8. The assay mixture was incubated for 30-90 minutes at 48° C.
9. The R3 reagent was injected into the main cuvette to adjust PH to >8.0 for fluorescence enhancement
10. Next T₁, fluorescence measurement was taken, and the result was calculated T₁/T ₀
11. Finally, the PHE concentration was calculated by using a calibration curve to translate the raw fluorescence data to a final PHE concentration.

Procedure 2. The golden standard reference blood metering and collection. After metering and adding the blood to the assay capsule all assay steps was performed by the Egoo device.

In procedure 2, the blood was collected using the standard blood spot (DBS) collection cards. Samples of defined areas of blood-filled membrane material were cut out (“stamped out” using a cutting device designed for the task). The cut (metered) membrane was inserted into the Egoo capsule device. All other assay steps were performed by the Egoo device. (During costumery procedure, after blood collection at home on the DBS collection cards, the cards were mailed to central laboratories at the hospital where trained personnel cut out defined areas thereby metering the sample).

Procedure 2 consisted of the following steps:
1. Full blood from a fingertip was added to the Whatman DBS blood collection card (Whatman 903 membrane), approx. 4×70 μl of blood is used, requiring intense massaging of the finger in order to produce the required amount of blood.
2. The filter was dried for 3 hours (during conventional use, this card was sent by the post to the hospital laboratory).
3. A defined membrane area was cut out of the blood containing membrane.
4. The blood containing membrane was inserted into the Egoo capsule and the Egoo device was used as described above in procedure 1 from step 5.

### Results

The following analytical performance characteristics tests were determined:

Precision
Intra precision study (intravariability)
Inter precision study (intervariability)

Blood samples containing approx. 50 μM and 500 μM phenylalanine (blood samples 1 and blood sample 2) were assayed in the two procedures.

Intra precision. The variation experienced by a single operator on a single device within a single series of PHE measurements (procedure 1 or procedure 2).

The results are shown in Table 3 below:

TABLE 3
Intra precision study for the PHE assay. Total of PHE 60
runs at the two concentrations using one Egoo device
n = 2 × (2 × 15) = 60 runs.

| | Procedure 1; n = 15 | | Procedure 2; n = 15 | |
|---|---|---|---|---|
| Egoo-9 | Mean dose | CV % | Mean dose | CV % |
| PHE blood | ~50 μM | 9.3 | ~50 μM | 21.1 |
| sample 1 | (sample 1) | | (sample 1) | |
| PHE blood | ~500 μM | 5.1 | ~500 μM | 11.3 |
| sample 2 | (sample 2) | | (sample 2) | |

Inter-precision study. Inter precision is the variation within a laboratory between days, different instruments and different operators. The results are shown in Table 4 below.

TABLE 4
Inter-precision between Procedure
1 and Procedure 2

| | Procedure 1 | | Procedure 2 | |
|---|---|---|---|---|
| Egoo-9 & 3 | Mean dose | CV % | Mean dose | CV % |
| PHE blood | ~50 μM | 9.9 | ~50 μM | 23.4 |
| sample 1 | (sample 1) | | (sample 1) | |
| PHE blood | ~500 μM | 6.7 | ~500 μM | 14.4 |
| sample 2 | (sample 2) | | (sample 2) | |

### Discussion

The purpose of the example 2 was to explore the possibility of integrating a well-known fluorescence-based PHE assay together with the described invention. The result indicates that the device based on the described invention is showing excellent performance comparable (or better) than the standard method using DBS collection cards.

As can be observed, the intra- and inter-precision using the present invention is significantly improved compared to collection and metering blood using the DBS collections cards following by the identical PHE assay on the Egoo device.

The reason for the significant better precision compared to the standard collection and metering method is likely more precise metering of the blood comparing the metering according to the invention with metering by cutting defined membrane areas (standard method).

Overall, it can be observed that in terms of precision, the PHE assay gave significant better assay results compared to the well-known standard DBS collection card method.

### Example 3. Precision of a Haemoglobin Assay Using the Present Invention Compared to Alternative Ways of Performing the Metering and Blood Collection Process

In example 1, five different method were used for metering and collection of the sample into the Egoo capsule. In example 3, the four best of those five methods (methods 2, 3, 4 and 5) were repeated using blood and a well-known haemoglobin assay. Each metering process was repeated 10 times using four methods and two Hb concentrations.

TABLE 5
Four metering and collection methods, method no 1 of example 1 is left out
due to very low precision data obtained in example 1.

| | |
|---|---|
| 2 (reference | 15 ul of blood sample material was collected and metered using the described |
| example) | disposable pipettes. The user collected the 15 μl sample by contacting the end of |
| | the pipette to the sample whereby 15 μl of the blood entered into the pipette by |
| | capillary forces. |
| | The entire sample was then added from the pipette directly to the detection liquid |
| | in the assay cuvette in an Egoo capsule by blocking the hole in the bulk part of the |
| | pipette with the fingers and applying pressure on the small bulk at the end of the |
| | sample filled pipette. The amount of haemoglobin was thereafter analysed. |
| 3 | 15 ul of blood sample material was collected and metered using the described |
| (embodiment | disposable pipettes. The user collected the 15 μl blood sample by contacting the |
| of the | end of the pipette to the blood sample whereby 15 μl of the blood entered into the |
| invention | pipette by capillary forces. |
| | The tip of the filled pipette was contacted to a membrane material whereby the |
| | entire sample flowed out of the pipette and into the membrane by passive capillary |
| | forces. The sample filled membrane was added to the assay cuvette in the Egoo |
| | capsule and the amount of haemoglobin was thereafter analysed. |
| 4 (reference | 15 ul of blood sample material was metered and directly added to a membrane |
| example- | material using a standard calibrated pipette (performed only by a laboratory |
| performed | technician). The blood sample filled membrane was added to the assay cuvette |
| only by a | in the Egoo capsule and the amount of haemoglobin was thereafter analysed. |
| laboratory | |
| technician) | |
| 5 (reference | 15ul of blood sample was metered and directly added to the assay cuvette in the |
| example- | Egoo capsule bypassing the collection membrane using a standard calibrated |
| performed | pipette (performed only by a laboratory technician) and the amount of haemoglobin |
| only by a | was thereafter analysed. |
| laboratory | |
| technician) | |

### Haemoglobin Assay Principle

Haemoglobin is a routine diagnostic parameter.

In this example, the well-known SLS haemoglobin detection method using cyanide-free sodium lauryl sulphate (SLS) was used. The reagent lyses red blood and white blood cells in the sample. The chemical reaction began by altering the globin and then oxidising the haeme group. Thereafter, the SLS' hydrophilic groups could bind to the haeme group and form a stable, coloured complex (SLS-HGB), which was analysed using a photometric method.

In the Egoo device, a LED (570 nm) sent out monochromatic light and by moving through the mixture light was absorbed by the SLS-HGB complexes. The absorbance was measured by a photo sensor and was proportional to the haemoglobin concentration of the sample.

### Summary of the Hb Assay Procedure

A precise volume of capillary blood (15 μl) was transferred from a fingertip to the blood metering transfer pipette. The blood metering transfer pipette was inserted into the capsule inlet and transferred to the filter by active process (reference methods 2 and 4) or passive transfer (method 3 according to the invention) or directly into the assay cuvette (reference method 5). Superior results in method 3 were observed when the blood was entering the membrane parallel to the fibers. Further, it was observed that superior results were observed when the membranes were closely stacked together. The blood-filled membrane was injected into the main cuvette (methods 2, 3 and 4) where the blood was instantly extracted out of the membrane using vortex movement. The Hb now formed SLS-HGB complexes that could be measured at 570 nm after 2 minutes incubation with the R1 reagent.

### Haemoglobin Assay Reagent

R4: Commercially available SLS haemoglobin detection reagent (Sysmex).

Assay procedure:
1. 15 μl of full blood from a finger stick was added to a blood metering transfer pipette
2. The metering transfer pipette was inserted into the inlet of the Egoo Hb capsule where the metering transfer pipette got into physical contact with a membrane material (methods 2, 3 and 4). The blood entered the membrane material by the 3 methods in Table 5. After the blood transfer, the metering transfer pipette was discarded
3. A sample blank measurement by measuring the absorbance to time T₀ was taken
4. After blood transfer to the membrane on R1, the blood was injected into the main cuvette, where the blood was mixed with the R4 reagent using an oscillating (vortex) movement. In method 5, the blood was added directly at this point
5. After 2 minutes, a T₁, absorbance measurement was taken, and the result was calculated T₁/T ₀.
6. Finally, Hb concentration was calculated by using a calibration curve to translate the raw absorbance data to a final Hb concentration.

### Analytical Performance Characteristics

Intra-precision. The intra-precision is the variability experienced by a single operator on a single device within a single series of haemoglobin measurements.

### Results

Results are shown in the Table 6. Intra-precision study for the Haemoglobin assay. 10 Hb assays were run using four methods and two Hb concentrations. A total of 4×2×10=80 Hb assay ran on Egoo.

TABLE 6
Precision % CV

| Mean dose | Method 2 | Method 3 | Method 4 | Method 5 |
|---|---|---|---|---|
| 4.0 mM | 11.4 | 1.9 | 2.1 | 2.0 |
| 10.0 mM | 10.8 | 2.4 | 3.5 | 3.4 |

Repeatability between method 1; method 2 and Method 4.

### Discussion

The purpose of example 3 was to explore the possibility of integrating a well-known absorbance-based Hb assay together with the present invention.

As can be observed from Table 6, the precision using the described invention (method 3) is comparable (or better) than procedures performed by a trained laboratory technician with a calibrated pipette. It can also be observed that applying any kind of active pressure to the transfer pipette resulted in significant increased % CV values (method 2).

1. -10. (canceled)

11. A method of measuring the amount of an analyte in a liquid sample, the method comprising the steps of:
a. providing a set of parts comprising:
i. a container for collecting a liquid sample material
ii. a filter material, and
iii. a detection device comprising a reaction liquid,
b. adding a metered amount of liquid sample material to the container,
c. transferring the metered amount of liquid sample material from the container to the filter material,
d. contacting the filter material containing the metered amount of sample material with the reaction liquid and mixing the reaction liquid and the filter material, thereby obtaining a detection liquid,
e. measuring the transmission of electromagnetic radiation at one or more wavelengths through the detection liquid and/or the emission of electromagnetic radiation at one or more wavelengths from the detection liquid,
f. detecting the amount of analyte in the sample by comparing the results obtained in step e. with an internal standard, wherein the metered amount of sample is transferred from the container to the filter material in step c by use of capillary forces.

12. The method according to claim 11, wherein filter material is capable of containing more liquid than the metered amount of liquid sample material added to the container in step b.

13. The method according to claim 11, wherein the blood sample is whole blood sample consisting of less than 50 μl, preferably less than 40 μl, even more preferably less than 40 μl.

14. The method according to claim 11, where the mixing in step d is performed by oscillating the detection liquid in a circular of ellipse motion.

15. The method according to claim 11 where the liquid comprises a substance which binds to an analyte present in the sample, such as the fluorophore eosin-borate-acid for HbA1 detection.

16. The method according to claim 11, where the analyte is phenylalanine, hs-CRP, HbA1c, Vitamin D, d-dimer or a lipid.

17. Kit-of-parts for performing the method according to claim 11.

18. Kit-of-parts comprising:
a. a container for collecting a metered amount of liquid sample material by capillary forces,
b. a filter material capable of collecting the metered amount of liquid sample material from the container by capillary forces, and
c. a detection device comprising a reaction liquid.

19. Kit of parts according to claim 18, further comprising:
a. a detection assembly comprising a source of electromagnetic radiation, means for detecting electromagnetic radiation, and means for receiving a detection device comprising a liquid sample,
b. a means for providing rapidly oscillations in a circular or ellipse motion of the detection assembly.
